(19) United States
(12) Patent Application Publication
(10) Pub. No.: US 2013/0157344 A1
(43) Pub. Date: Jun. 20, 2013

(54) METHOD FOR PRODUCING OIL-RICH MICROALGAE AS FEEDSTOCK FOR BIODIESEL PRODUCTION

(71) Applicants: Fan Lu, Clemmons, NC (US); Zhongyang Deng, Winston-Salem, NC (US)

(72) Inventors: Fan Lu, Clemmons, NC (US); Zhongyang Deng, Winston-Salem, NC (US)

(73) Assignee: CHLOR BIOENERGY INC., Jersey City, NJ (US)

(21) Appl. No.: 13/704,489

(22) PCT Filed: Jun. 14, 2011
(86) PCT No.: PCT/US11/40268

§ 371 Date: Feb. 26, 2013

Related U.S. Application Data
(60) Provisional application No. 61/354,485, filed on Jun. 14, 2010.

(51) Int. Cl. C12P 7/64

(52) U.S. Cl. CPC C12P 7/649
USPC 435/257.1

(57) ABSTRACT

A process for production of oil-rich microalgae is disclosed, which includes methods for cultivating strains of Characium polymorphum and/or Ankistrodesmus braunii (Chlorophyceae), or isolated variants thereof, in order to produce oil at optimum levels. The process is suitable for large-scale productions. The invention also discloses methods for purifying the microalgae cells, and methods for treating the microalgae cells to enrich their oil content. In addition, various representative culturing media as well as conditions for cultivating microalgae and inducing oil accumulation are also disclosed. The oil-rich microalgae produced by the process can be used as feedstock for biofuel production.

### CROSS-REFERENCE TO RELATED APPLICATIONS

This application claims priority under 35 U.S.C. §119(e) to U.S. Provisional Patent Application Ser. No. 61/354,485, filed on Jun. 14, 2010, which is hereby incorporated by reference in its entirety for all purposes.

### FIELD OF THE INVENTION

The present invention relates to culturing media and processes for production of oil-rich microalgae, which can be used as feedstock for biodiesel production. The process is suitable for large-scale production owing to the unique microalgal species used.

### BACKGROUND OF THE INVENTION

The United States spends $100-150 billion each year for 60 billion gallons of petroleum diesel and 120 billion gallons of gasoline to drive vehicles on the road (U.S. Department of Energy statistics). Most of these fossil fuels are imported from countries, where political instability, human rights abuses, and terrorism are a constant threat to a stable oil supply. Heavy dependence on foreign oil has not only put the U.S. in a strategic weak position, but also contributes to U.S. trade deficit, adding constrains to the national economy. Moreover, it is well established that carbon dioxide emission from various petroleum powered transportation systems accounts for one third of the total carbon dioxide released into the atmosphere, leading to climate change. The toxic exhaust gas from petroleum-powered vehicles also causes hazardous effects on human health.

To address these problems associated with fossil fuel, cleaner and more reliable alternative sources of oil have to be developed before the exhaustion of the remaining known oil reserves within the next 50 years or so. In fact, intensive research and planning for alternative fuels has been undertaken since more and more governments have recognized that a problem exists. Among the possible alternatives, biodiesel appears to be the most promising fuel to power automobiles in the future.

Biodiesel, derived from vegetable oils or animal fats rather than crude oil, is an alternative fuel for diesel engines. It is renewable, non-toxic, and biodegradable. It can be used in existing diesel engines without modification, and can be blended in any ratio with petroleum diesel. Currently, the major feedstock for biodiesel production is soybean oil. However, low oil production rates (ca. 1-3 barrels of oil per acre of land per year) coupled with high production costs (e.g. feedstock accounts for 70-80% of biodiesel production costs) have limited the expansion of soybean-based biodiesel to meet the growing demand by society. Therefore, it is important to develop more economically viable sources of feedstock for biodiesel production.

Microalgae are known to exhibit 10- to 20-fold higher growth rates than agricultural crop plants, and certain microalgal species can accumulate large amounts of lipids or oil (30-60% of dry weight). As a result, the concept of using microalgae as an alternative source of feedstock for biodiesel production was intensively studied through the ‘Aquatic Species Program’ supported by the U.S. Department of Energy from 1978 to 1996 (A Look Back at the U.S. Department of Energy's Aquatic Species Program: Biodiesel from Algae. Close-Out Report, NREL/TP-580-24190). The project was focusing on selection of suitable microalgal species, manipulation of microalgal metabolism, and tests on microalgal production systems.

However, a conclusion from the Aquatic Species Program was that microalgae-based biodiesel was not economically viable because of high production cost. This conclusion was mainly based on studies using open raceway ponds, the only culture system tested at that time. that the open raceway ponds suffer seriously from several critical drawbacks, including lack of control of culture temperature, light intensity and contamination. The failure to develop a commercially viable microalgae-based biodiesel production system is largely attributable to the lack of cost-effective and efficient photobioreactors during the ‘Aquatic Species Program’. Therefore, there is a clear need to develop new culture systems and methods for more efficient and economic production of oil from microalgae, especially in large scales.

### SUMMARY OF THE INVENTION

The present invention meets the foregoing need by providing a new method for large-scale production of oil-rich microalgae. Specifically, the present invention provides use of cells and/or strains of microalgae Characium polymorphum and/or Ankistrodesmus braunii (Chlorophyceae) for oil production and methods for optimizing oil production within microalgae Characium polymorphum and/or Ankistrodesmus braunii.

More specifically, this invention provides culture media and conditions used for optimizing oil production in microalgae Characium polymorphum and/or Ankistrodesmus braunii.

In one aspect the present invention provides a process for production of oil-rich microalgae, the process comprising: a) purifying microalgae; b) cultivating the microalgae in a culture medium; and c) inducing oil accumulation in the microalgae.

In another aspect the present invention provides a culture medium used for production of oil-rich microalgae, the culture medium comprises about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients. In one embodiment the culture medium further comprises about 0.1-2 g/L NaNO₃.

In another aspect the present invention also encompasses use of cells or strains of Characium polymorphum, Ankistrodesmus braunii, or isolated varieties or combinations thereof, in production of oil-rich microalgae or biodiesel.

In another aspect the present invention encompasses use of the processes described herein for producing oil-rich microalgae useful as feedstock for biodiesel production.

These and other aspects of the present invention will be better appreciated by reference to the following drawings, detailed description, and claims.

### BRIEF DESCRIPTION OF THE DRAWINGS

FIG. 1 illustrates the effects of Hight Light (200 μmol m⁻² s⁻¹), Low Light (50 μmol m⁻² s⁻¹), and Dark on the growth of Characium polymorphum. Cells were inoculated in full medium as described in Example 2). Cells were withdrawn every two days to monitor the changes in dry weight.

FIG. 2 illustrates the effects of light intensity on lipids contents of Characium polymorphum. Cells were inoculated in nitrogen-free medium and were exposed to different light intensity for 10 days. Total lipid contents were measured as percentage of cellular dry weight.

### DETAILED DESCRIPTION OF THE INVENTION

In one aspect the present invention provides a method for purifying microalgae, the method comprising: washing microalgae cells with a sterile medium; spreading the microalgae cells onto an agar plate containing a growth medium; illuminating the microalgae cells with a light for a period of time.

In one embodiment, the method further comprises transferring the microalgae cells from the agar plate containing the growth medium to a fresh plate.

In one embodiment, said illuminating period is from about three (3) days to about one month (30 days), although a shorter or longer time is also contemplated.

In a preferred embodiment, the microalgae cells are illuminated for a period of from about 5 days to 15 days.

In another preferred embodiment, the microalgae cells are illuminated for one week (7 days) to 10 days.

In another embodiment, said transferring is repeated until axenic colonies are obtained. Preferably, said transferring is repeated from 1 to 5 times. More preferably, said transferring is repeated for 2 or 3 times.

In another embodiment, the method further comprises collecting the axenic colonies for further cultivation.

In another embodiment, the method further comprises cultivating the cultured microalgae cells as described above until optimum oil production is achieved. In one preferred embodiment, said optimum oil production means that the microalgae contains about 40 to 50% oil.

In a preferred embodiment, the growth medium comprises about 0.1-2 g/L NaNO₃, about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients. The term “EDTA” stands for ethylenediaminetetraacetic acid.

In another preferred embodiment, the light for illuminating is a fluorescent light.

A person of ordinary skill in the art would be able to use or modify the method described to enhance oil production by such algae until an optimum result is achieved. Thus, any variants or equivalents of the method described above are also encompassed by the present invention.

In another aspect the present invention provides a culture medium for optimal growth of oil-rich microalgae.

In one embodiment, the culture medium comprises about 0.1-2 g/L NaNO₃, about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients.

In a preferred embodiment, the culture medium consists essentially of about 0.1-2 g/L NaNO₃, about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients.

In a more preferred embodiment, the culture medium consists essentially of 0.1-2 g/L NaNO₃, 0.05-1.75 g/L MgSO₄, 0.5-3.6 g/L Na₂CO₃, 0.05-0.2 g/L CaCl₂, about 0.001 g/L EDTA, 0.02-1.2 g/L K₂HPO₄, about 0.006 g/L Citric Acid, about 0.006 g/L Fe(NH₄)₂Citric, and 0.2-1 ml/L A5 micronutrients

In another aspect the present invention comprises a method of cultivating microalgae cells, comprising the steps of:

a) maintaining microalgae cells in a growth medium for a period of time;

b) cultivating the microalgae cells in a first reactor containing a first volume of medium for a period of time;

c) cultivating the microalgae cells in a second reactor containing a second volume of medium for a period of time; and

d) cultivating the microalgae cells in a third reactor illuminating with a light.

In one embodiment, said maintaining step comprises maintaining the microalgae cells in an agar plate containing the growth medium, preferably for at least about 120 hours.

In another embodiment, said first reactor is a 100 mL or flask, and said volume of medium is about 50 mL, and said period of time is at least about 72 hours.

In another embodiment, said second reactor is a 2 L flask, and said volume of medium is about one liter (1 L), and said period of time is at least about 72 hours.

In another embodiment, said third reactor is a photobioreactor, which is preferably 5 liter or greater column photobioreactor or 20 liter or greater flat photobioreactor.

In another embodiment, the light intensity for illuminating is in the range of from about 10 to about 2500 μmol m⁻² s⁻¹.

In another preferred embodiment, the temperature for cultivating is from about 5° C. to about 40° C.

In another preferred embodiment, carbon dioxide is provided to the cell culture as a mixture with air at a concentration from about 0.1% v/v to about 10% v/v. The sequence of cultivation steps set forth above represents a preferred embodiment, but the exact sequence is not required. Therefore, the invention encompasses any combinations of the steps in any orders.

In another aspect the present invention provides a method of enhancing oil production by microalgae Characium polymorphum or Ankistrodesmus braunii strains, the method comprising the steps of:

a) transferring cells of the microalgae Characium polymorphum and/or Ankistrodesmus braunii from a bioreactor to a nitrogen-deficient medium;

b) maintaining the cells in a nitrogen-deficient medium at a pre-determined temperature for a period of time; and

c) illuminating the microalgae cells with a light.

In one embodiment of this aspect, the nitrogen-deficient medium comprises about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients.

In a preferred embodiment, the nitrogen-deficient medium consists essentially of about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients.

In a more preferred embodiment, the nitrogen-deficient medium consists essentially of 0.05-1.75 g/L MgSO₄, 0.5-3.6 g/L Na₂CO₃, 0.05-0.2 g/L CaCl₂, about 0.001 g/L EDTA, 0.02-1.2 g/L K₂HPO₄, about 0.006 g/L Citric Acid, about 0.006 g/L Fe(NH₄)₂Citric, and 0.2-1 ml/L A5 micronutrients.

The temperature for maintaining the microalgae cells in the nitrogen-deficient medium can be in the range from about 1 to about 35° C. As a person of ordinary skill in the art would understand, the higher temperature, the faster for the microalgae to accumulate certain content of oil. To illustrate, for example, at about 25 to about 30° C., oil production in the microalgae can be enhanced to 40%-50% of dried weight after 5-20 days in the nitrogen deficient medium. At a lower temperature from about 1 to about 15° C., oil production can be enhanced to 40-50% of dried weight by the microalgae after about 10 to about 30 days.

In a preferred embodiment, the intensity of the light used to illuminate the microalgae is higher than 200 μmol m⁻² s⁻¹. The light can be generated from any light sources.

Preferably, the light is used to enhance the oil production until it reaches about 40%-50% of dried weight. This can take about 5-30 days depending on other conditions.

In another preferred embodiment, the invention provides a method to induce oil accumulation in the microalgae cells by a combination of (a) maintaining the cells in a nitrogen deficient medium and (b) illuminating the cells with a light.

In one embodiment of the combination conditions, the nitrogen-deficient medium comprises about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients; and the light intensity is higher than 200 μmol m⁻² s⁻¹.

In a preferred embodiment, the nitrogen-deficient medium consists essentially of about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/L A5 micronutrients; and the light intensity is higher than 200 μmol m⁻² s⁻¹. Under these conditions, the oil content in cells reaches 40-50% in less than 5 days.

In another aspect the present invention also encompasses use of cells or strains of Characium polymorphum, Ankistrodesmus braunii, or isolated varieties or combinations thereof, in production of oil-rich microalgae or biodiesel.

In another aspect the present invention encompasses use of the processes described herein for producing oil-rich microalgae useful as feedstock for biodiesel production.

The term “about,” as used herein, refers to a range of values within ten percent (10%) of a baseline value. Thus, for example, the phrase “about 100” refers to a range of values between 90 and 110.

When the term “about” is applied to a range, it indicates that both the upper limit and lower limit can vary up to ten percent (10%) of the base line value.

The term “a,” “an,” or “the,” as used herein, represents both singular and plural forms. In general, when either a singular or a plural form of a noun is used, it denotes both singular and plural forms of the noun.

The term “biodiesel,” as used herein, refers to commonly known fatty acid esters (e.g., methyl, ethyl, propyl, etc.). The microalgae produced according to the present invention contains mainly lipids (including oil—triglycerides, free fatty acids, phospholipids, and so on). After cultivation of the microalgae, a microalgal biomass is first harvested, which is then extracted to obtain lipids (oil and other lipids). These lipids will be used as feedstock for biodiesel production. For example, the lipids extracted from the oil-rich microalgae can be readily converted to biodiesel by known chemical reactions and/or chemical engineering processes (e.g., by esterification and/or transesterification).

The biodiesel produced from the oil-rich microalgae of the present invention has wide applications, which include, but are not limited to, use in standard diesel engines or converted diesel engines of vehicles, trains, aircrafts, etc., or use as heating fuel in either domestic or commercial boilers. The biodiesel can be used alone or blended with petroleum diesel. It can also be used as a low carbon alternative to heating oil. Such uses or variants of uses are within the knowledge of a person of ordinary skill in the art, description of which is merely for illustration purpose, but not intended to be limiting.

The invention will be further illustrated by the following non-limiting examples.

### EXAMPLES

### Example 1

### Purification of Microalgae

After washing with sterile medium, microalgae cells were spread onto agar plates containing respective growth media (see 2. below). The plates were illuminated with fluorescent light. After one week, microalgae cells from the plates were transferred to a fresh plate. After three transfers, axenic colonies were obtained, which were picked up for further cultivation.

### Example 2

### Cultivation of Microalgae Cells

The following steps are used for cultivation of the microalgae cells:

(a) Maintenance of the cells in agar plates containing growth medium for at least about 120 hours;

(b) Cultivation of the cells in 100-ml flasks containing 50 ml medium for at least about 72 hours;

(c) Cultivation of the cells in 2-liter flasks containing 1 liter medium for at least about 72 hours; and

(d) Cultivation of the said strains in 5-liter column photobioreactors and 20-liter flat-plate photobioreactors.

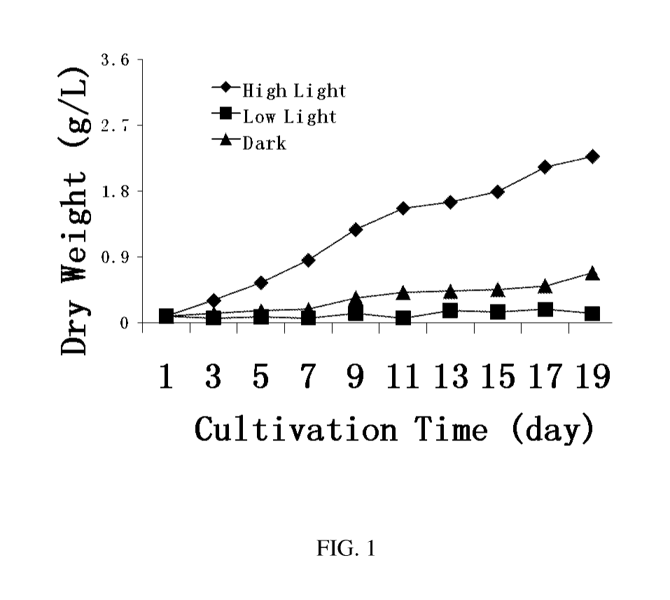

The light intensity is between 10-2500 μmol m⁻² s⁻¹ (see, e.g., FIG. 1), and the temperature is between 5° C. and 40° C. Carbon dioxide is provided to the cell culture as a mixture with air at a concentration of 0.1%-10% (v/v). It is preferable (but not required) to carry out the cultivation steps described in the sequence set forth above.

### Example 3

### Enhancement of Oil Production by the Microalgae

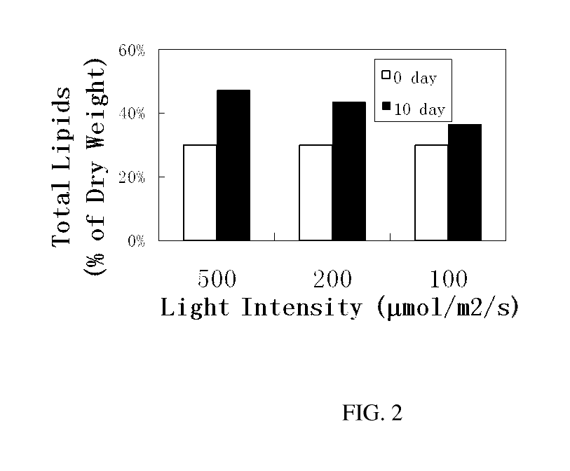

The following steps are used for enhancement of oil production by Microalgae:
(a) Transfer the cells of the strains from the flat plate bioreactor to a nitrogen-deficient medium, containing:
   0.05-1.75 g/L MgSO₄
   0.5-3.6 g/L Na₂CO₃
   0.05-0.2 g/L CaCl₂
   0.001 g/L EDTA
   0.02-1.2 g/L K₂HPO₄
   0.006 g/L Citric Acid
   0.006 g/L Fe(NH₄)₂Citric
   0.2-1 ml/L A5 micronutrients
The cells are maintained in this medium at a temperature range of 25-30° C. for at least 7 days. Using this technique, oil production in the microalgae can be significantly enhanced to 40%-50% of dried weight after 5-20 days in the nitrogen deficient medium.
(b) In an alternative embodiment exposing the microalgae cells to a low temperature (1-15° C.) environment can also enhance oil production to 40%-50% of dried weight by the said microalgae after 10-30 days.
(c) The microalgae cells are exposed to a light intensity higher than 200 μmol m⁻²-s⁻¹ from any light source to enhance the oil production to 40%-50% of dried weight by the microalgae after 5-14 days (see, e.g., FIG. 2).
(d) One preferred method to induce oil accumulation in the said microalgae cells is the combination of maintaining the cells in a nitrogen deficient medium (as set forth above) and exposing the cells to the light intensity higher than 200 μmol m⁻² s⁻¹. Under these conditions, the oil content in cells reaches 40-50% in less than 5 days.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A process for production of oil-rich microalgae, the process comprising: a) purifying microalgae selected from the group consisting of Characium polymorphum, Ankistrodesmus braunii, isolated varieties and combinations thereof; b) cultivating the microalgae in a culture medium; and c) inducing oil accumulation in the microalgae.

2. The process according to claim 1, wherein said microalgae comprise cells or strains of Characium polymorphum or isolated varieties thereof.

3. The process according to claim 1, wherein said microalgae comprise cells or strains of Ankistrodesmus braunii or isolated varieties thereof.

4. The process according to claim 1, wherein said microalgae comprise cells or strains of Characium polymorphum and Ankistrodesmus braunii, or isolated varieties thereof.

5. The process according to claim 1, wherein said culture medium comprises about 0.1-2.0 g/L NaNO₃, about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1.0 ml/liter A5 micronutrients.

6. The process according to claim 1, wherein said culture medium consists essentially of about 0.1-2.0 g/L NaNO₃, about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1.0 ml/liter A5 micronutrients.

7. The process according to claim 1, wherein said purifying comprises transferring the microalgae to agar plates containing said culture medium.

8. The process according to claim 1, further comprising scaling up the culture of the microalgae in a photobioreactor.

9. The process according to claim 8, wherein said scaling up comprises:
maintaining the microalgae in agar plates containing a growth medium; culturing the microalgae in a reactor containing 50-1000 mL medium; and culturing the microalgae in 5-20 L photobioreactors.

10. The process according to claim 1, wherein said cultivating is conducted at a light intensity in the range of from about 10 μmol m⁻²-s¹ to about 2500 μmol m⁻² s⁻¹, at a temperature in the range from about 5° C. to about 40° C.; and at a carbon dioxide concentration in the range from about 0.1% v/v to about 10% v/v.

11. The process according to claim 1, wherein said culture medium comprises about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/liter A5 micronutrients.

12. The process according to claim 1, wherein said inducing oil accumulation comprises cultivating cells of microalgae at a temperature from about 1° C. to about 30° C.

13. The process according to claim 1, wherein said inducing oil accumulation comprises cultivating cells of microalgae at a temperature from about 25° C. to about 30° C.

14. The process according to claim 1, wherein said inducing oil accumulation comprises transferring cells of the microalgae in the presence of a light having an intensity above 200 μmol m⁻² s¹.

15. The process according to claim 1, further comprising incubating microalgae cells in a nitrogen deficient medium and exposing the cells to a light.

16. The process according to claim 15, wherein said light has an intensity of at least 200 μmol m⁻² s⁻¹.

17. A culture medium for enhancing oil accumulation in microalgae, comprising about 0.05-1.75 g/L MgSO₄, about 0.5-3.6 g/L Na₂CO₃, about 0.05-0.2 g/L CaCl₂, about 0.001 g/L EDTA, about 0.02-1.2 g/L K₂HPO₄, about 0.006 g/L Citric Acid, about 0.006 g/L Fe(NH₄)₂Citric, and about 0.2-1 ml/liter A5 micronutrients.

18. The culture medium for enhancing oil accumulation in microalgae according to claim 17, consisting essentially of 0.05-1.75 g/L MgSO₄, 0.5-3.6 g/L Na₂CO₃, 0.05-0.2 g/L CaCl₂, about 0.001 g/L EDTA, 0.02-1.2 g/L K₂HPO₄, about 0.006 g/L Citric Acid, about 0.006 g/L Fe(NH₄)₂Citric, and 0.2-1.0 mL/liter A5 micronutrients.

19. The culture medium for enhancing oil accumulation in microalgae according to claim 17, further comprising about 0.1-2 g/L NaNO₃.

20. The culture medium for enhancing oil accumulation in microalgae according to claim 17, consisting essentially of 0.1-2 g/L NaNO₃, 0.05-1.75 g/L MgSO₄, 0.5-3.6 g/L Na₂CO₃, 0.05-0.2 g/L CaCl₂, about 0.001 g/L EDTA, 0.02-1.2 g/L K₂HPO₄, about 0.006 g/L Citric Acid, about 0.006 g/L Fe(NH₄)₂Citric, and 0.2-1 ml/liter A5 micronutrients.

21. Use of cells or strains of Characium polymorphum, Ankistrodesmus braunii, or isolated varieties or combinations thereof, in production of oil-rich microalgae useful as feedstock for production of biodiesel.

22. (canceled)
